(19) United States
(12) Patent Application Publication
(10) Pub. No.: US 2015/0072376 A1
(43) Pub. Date: Mar. 12, 2015

(54) TRYPTOPHAN AS THE FINGERPRINT FOR DISTINGUISHING AGRESSIVENESS AMONG CANCER CELL LINES USING NATIVE FLUORESCENCE SPECTROSCOPY

(71) Applicant: Robert R. Alfano, Bronx, NY (US)

(72) Inventors: Robert R. Alfano, Bronx, NY (US); Lin Zhang, Brooklyn, NY (US); Yang Pu, New York, NY (US)

(21) Appl. No.: 14/485,353

(22) Filed: Sep. 12, 2014

Related U.S. Application Data
(60) Provisional application No. 61/960,184, filed on Sep. 12, 2013.

(51) Int. Cl. G01N 21/64; G01N 33/483

(52) U.S. Cl. CPC G01N 21/6486; G01N 33/4833; G01N 2800/7028
USPC 435/34; 435/288.7

(57) ABSTRACT

Tryptophan is used as the key native marker in cells to determine the level of aggressiveness of cancer cell lines using the native fluorescence spectroscopy. A ratio R of the fluorescence from tryptophan at 340 nm to that from the NADH at 440-460 nm is demonstrated to be associated with aggressiveness of the cancer cells. The higher the ratio R, the more aggressive the tumor towards metastasis.

### BACKGROUND OF THE INVENTION

The ability of cancer tumors to metastasize is an ominous feature of malignant tumors. Metastasis is the primary cause of death among cancer patients. One third of the people will receive a diagnostic of cancer during one's life, and one third of them will die of this cancer due to metastasis. In 2007, about 123 million cancer cases and 7.6 million cancer deaths are estimated to have occurred, which is the second most common cause of death only after heart disease; moreover, breast cancer is the leading cause of cancer death among females(1). Methods to determine aggressive cancer in situ has been studied throughout the world.

One promising method to diagnose cancers tissue without removing tissue is based on optical spectroscopy (2, 3). The field using optical spectroscopy on biomedical samples has been coined: ‘Optical Biopsy’ which is becoming commonplace to determine the state of tissue in vivo and ex viva The major focus in Optical Biopsy is to measures native fluorescence (NFL) (2, 3) to characterize the properties of normal, benign and malignant metastasis cancers in tissues and cells. The main fluorophores in tissues and cells include tryptophan, collagen, elastin, reduced nicotinamide adenine dinucleotide (NADH), porphyrins, and flavin adenine dinucleotide (FAD). For cells there is no collagen, elastin and porphyrins. They may appear in different content due to tumor evolution. These changes can be reflected by the NFL spectral fingerprints with distinct excitation and emission spectra maxima or peaks. These key intrinsic molecules in cells and tissues have unique spectral profiles for absorption and emission from the ultraviolet (UV) to visible range. The emission from tryptophan is clearly the main fluorescence over the others molecules upon exciting the tissues and cells using light at approximately ≦300 nm. Tryptophan is an essential amino acid needed to synthesize proteins and locate in the cells. Tryptophan is the ‘food’ not only for cancer cells, but also for immune cells, the more cancer cells consume, the less is left for immune cells. Starvation of the immune cells causes apoptosis, the immune system fails to detect the cancer cells and the cancer cells can spread easily.

The invention teaches that tryptophan level is an important biomarker for determining aggressive cancers in cells. NFL spectroscopy is used as an effective approach to distinguish cancer cell lines with different metastatic ability as well as normal cell lines, based on their tryptophan levels. Upon excitation at approximately 300 nm, the ratio of the emission peak at 340 nm from tryptophan and that from NADH at 440-460 nm and flavins of 500-525 nm was measured and calculated from various breast cell lines. The experiment and analysis results indicate and teach that the relative content of tryptophan reflected by NFL can be used to determine the aggressive nature of cancer growth to other parts of the body. Also, this applies to prostate and other cell lines from cervix, colon, bladder, stomach, brain, kidney, liver, oral etc.

### SUMMARY OF THE INVENTION

An object of the present invention is to focus on providing a novel optical approach for monitoring the metastasis competence of cancerous cells using native fluorescence spectroscopy, which is quick and easy. The main fingerprint is the emission at ˜340 nm emitted from tryptophan. FIG. 1 shows the native fluorescence emission spectra of aggressive cancerous cells (MDA-MB-231), non-aggressive cancerous cells (MCF-7) and normal cells (fibroblast cells) deviate and emission intensifies at selected wavelengths are compared. The metastasis competence is classified by the cell provider.

It is also an object of the present invention to provide a method and system, as described above, that overcome at least some of the problems associated with previous conventional methods proposed to detect metastasis competence of cancer, such as bone marrow examination, pathologic slices from biopsy etc.

It is another objective of the present invention to provide an optical method, which will be easily developed to compact commercialized device to observe the therapeutic effect on cancer.

It is still another object of the present invention to provide a method and system, as described above, that have extended applications for the real-time monitoring the cancer development or progress.

In accordance with the above objectives, as well as with those objects to become apparent from the description to follow, there is hereinafter disclosed a novel method and system for detecting metastasis competence and monitoring the therapeutic effect on cancer, by using a spectrofluorometer system, with excitation wavelengths, said methods and system employing an illuminating light beam output for lasers or light emitting diodes (LEDs) with selective wavelength, optical components (band pass filters, pass long filters and optical fibers), probes, and optical detectors (compact CCD spectrometer and fiber spectrometer).

The ratios R of intensities at 340 nm for tryptophan over 460 nm for NADH were calculated for each sample and compared the differences in FIG. 2. The average ratio of all aggressive cancerous cells was 11.2, while the average ratio for non-aggressive cells was 5.98 and the average ratio for normal cells was 4.36. The average ratios are R₂₃₁>RM_{CF-7}>R_{Normal}. The differences of the ratios for 340 nm over 460 nm are prominently higher in aggressive cancerous cells compared to non-aggressive cancerous cells as well as normal cells. There is good agreement with our past observations on breast tissue which used the ratio of 340 nm to 440 nm in some cancer tissues with ratios above 10, even up to 50. In addition, these features were demonstrated for aggressive prostate cancer cell.

Moreover, the increase in the average ratio R from non-aggressive to aggressive cancerous cells, and from normal cells to non-aggressive cancerous cells provides a diagnostic criterion for detecting metastasis competence level. In order to evaluate this potential, FIG. 2 shows the calculated average ratio R (box) of the ratios as a function of normal, non-aggressive and aggressive cancerous cells. It is important to note that the average R values exhibit a linear dependent property of monotonous growth and provide a good correlation with the metastasis competence. The higher the average ratio of R indicates the higher the competence to metastasis. In cancer diagnosis and cancer research, identification of the metastatic competence of cancer is crucial to determine the stage and therapeutic method. This study provides a highly relevant methodology to predict the metastatic potential of cancers by measuring the tryptophan spectra (4).

The present invention is based on the contents changes of the tryptophan in the aggressive cancerous cells, non-aggressive cancerous cells and normal cells. The measurement results indicate that there is a higher tryptophan content in aggressive cancerous cells, which accords to the physiology phenomena in cancer. Tryptophan (or L-tryptophan) is an essential amino acid in the cytoplasm of the cells, act as building blocks in protein biosynthesis. It can't be synthesized by mammalian cells and therefore must be part of diet. Tryptophan is transported into cancer cells via large amino acid transporter system (LAT1/CD98) and is processed into several components production, e.g, (1) sertonin (a neurotransmitter in central nervous systems), (2) niacin (known as Vitamin B3, niacin deficiency will associate with pellagra), (3) tryptophan can also be metabolized into kynurenine by the enzyme IDO (ndoleamine-2 3-dioxygenase), kynurenine can dilate blood vessels during inflammation and regulate immune response. Tryptophan is the ‘food’ not only for cancer cells, but also for immune cells, the more cancer cells consume, the less there is left for immune cells. Numerous studies have indicated that the tryptophan consumption by cancer cells in suppresses the immune response to cancer cells. The immune system T cells are particularly susceptible to low tryptophan concentrations and result in anergy and apoptosis, so that cancer cells can escape immune detection and survive. An increasing number of studies show that the fast progress of tumor is due to a failure of immune system control over the growth of tumor cells (5, 6). In above T cells ‘death by starvation’ paradigm, the cancer cells escape immune detection from T cells and develop towards increasingly aggressive forms. Therefore, direct monitoring of the tryptophan level in cells/tissue can be used key to investigate or monitor the immune escaping ability of the cancer cells and the metastasis ability of mild and aggressive breast and other cancer cells in the prostate.

During a diagnosis, one may determine if the result is positive (disease) or negative (healthy). It is necessary to eliminate statistical errors, such as false positive or false negative. False positive is defined as a test result that is erroneously classified in a positive category and false negative is defined as a test result that is erroneously classified in a negative category. To set the diagnosis standard for distinguishing the positive or negative results and evaluating the metastasis level of the cancer, a Support Vector Machine (SVM) was applied, one of the most useful technique for data classification to categorize the three groups of data, on all 51 (3 types) cell samples. In general, the SVM classifier is determined by a number of components for most effectively discriminating the support vectors located at the boundary of the group of data. In this case, since the data is just in one dimension, a Support Component Machine (SCM) (a simplified SVM for one dimension) and Support Component (SC) can be used instead of SVM and Support Vector (SV). To distinguish aggressive, non-aggressive and normal cells, the SCs are chosen from the components of R (the ratios) between

Rₘᵢₙ^{agg}−κ and Rₘₐₓ^{non-agg}−κ,

where κ is a self-defined threshold value for optimum, and

Rₘᵢₙ^{agg} and Rₘₐₓ^{non-agg}

is the minimal R for aggressive and maximal R for nonaggressive cells. The same method was applied for chosen SCs for identifying cancer cells of both aggressive and non-aggressive from normal cells.

It was found that the criteria R=5.27 and R=6.77 distinguish normal vs. cancer, and non-aggressive vs. aggressive, respectively, and in aggressive cancer cells the average ratio R=112, which is shown as solid lines in FIG. 3. According to these two separating lines, the sensitivity and specificity for these two cases can be calculated.

The receiver operating characteristic (ROC) curves were used to evaluate the performance of criterion of the calculated R of the 340 over 460 nm ratio combined with SCM for distinguishing the metastasis competence of cancerous cells as well as normal cells. Accuracy can be measured by the AUC (area under the ROC curve). The ROC curves shown in FIGS. 4 and 5 were generated from the cases of aggressive vs. non-aggressive cells, and cancer vs. normal cells, respectively, to determine the accuracy of metastasis levels by using the ratio R, combined with SCM. The AUC values of the ROC curves were then calculated to evaluate the accuracy.

The sensitivity, specificity and the AUC values for using the calculated ratios combined with SCM for detecting cancerous cells metastasis competence are summarized in Table 1. In Table 1, the excellent sensitivity, specificity and AUC values demonstrate the excellent efficacy using the ratios combined with SCM for separating different metastasis competence cancerous cells as a promising diagnostic tool for early cancer detection.

TABLE 1
Evaluation of performance for criterion using ratios of 340 nm over
460 nm combined with SCM for separating different type
of cancerous cells.

| Evaluated Components | Sensitivity | Specificity | AUC |
|---|---|---|---|
| Normal vs. Cancerous cells | 91.4% | 81.3% | 0.96 |
| Non-aggressive vs. Aggressive | 88.9% | 90.9% | 0.97 |
| Cancerous cells | | | |

NFL spectra have been used in the diagnosis of the aggressiveness of breast cancer. This is the first time, however, that cancer metastasis competence among aggressive cancer cells, non-aggressive cancer cells and normal cells has been evaluated by measuring the relative contents of tryptophan using NFL. Our results demonstrate aggressive breast cancer cells have higher relative contents of tryptophan than other cells. Tryptophan is a marker for aggressiveness of cancer cells using the ratio of 340 to 460 nm excited by 300 nm. This research indicates that measuring the NFL of tryptophan within the tissue cells can be used as a fingerprint for monitoring different metastasis cancers. A similar outcome was found in prostate cells supporting the results presented here. This technique may have potential to be used to monitor disease activity and response to therapy in cancer patients. From this research one can speculate that the size of the ratio R from tryptophan in cells indicates the degree on how aggressive the cancer is to metastasize, i.e. the Kiger the R>12 the more aggressive the cancer. Cells can be extracted from the organ (breast, prostate, brain, colon . . . ) and tested by R at 340/460 nm NSF to determine the level of the aggressiveness of the tumor.

### BRIEF DESCRIPTION OF THE FIGURES

FIG. 1 illustrates key wavelengths of absorption spectra of tissue fluorophore components;

FIG. 2 illustrates key wavelengths of emission spectra of tissue fluorophore components;

FIG. 3 illustrates the Native fluorescence emission spectra of all three types of cells with standard deviation error bars. MDA-MB-231 (solid line), MCF-7 (dash line) and Fibroblast (dotted line) excited at 300 nm wavelength;

FIG. 4 illustrates the increases of the average ratios R of 340 nm over 460 nm as a function of normal cells (Fibroblast), non-aggressive cancerous cells (MCF-7) and aggressive cancerous cells (MDA-MB-231); and

FIGS. 5-7 show test results, FIG. 5 illustrating the average ratios R, for 340 nm over 460 nm of total 3 types of cells. The separating lines were calculated using SCM. Accuracy evaluated by ROC curve using the extracted R for distinguishing significant criteria to classify the cell samples into two groups for cancer vs. normal cells (FIG. 4) and aggressive vs. non-aggressive cancerous cells (FIG. 5). One can use these methods in different cell types.

### DETAILED DESCRIPTION OF PREFERRED EMBODIMENTS

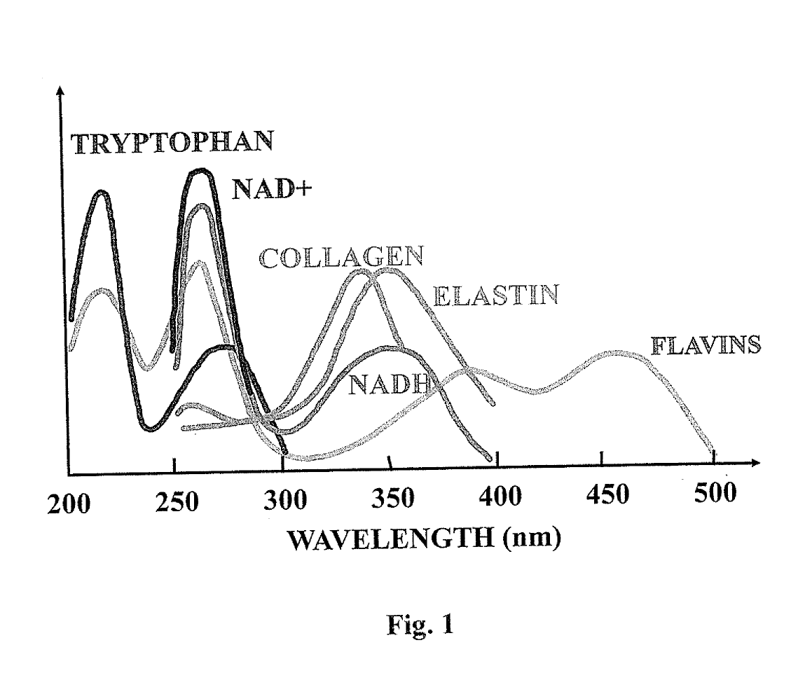

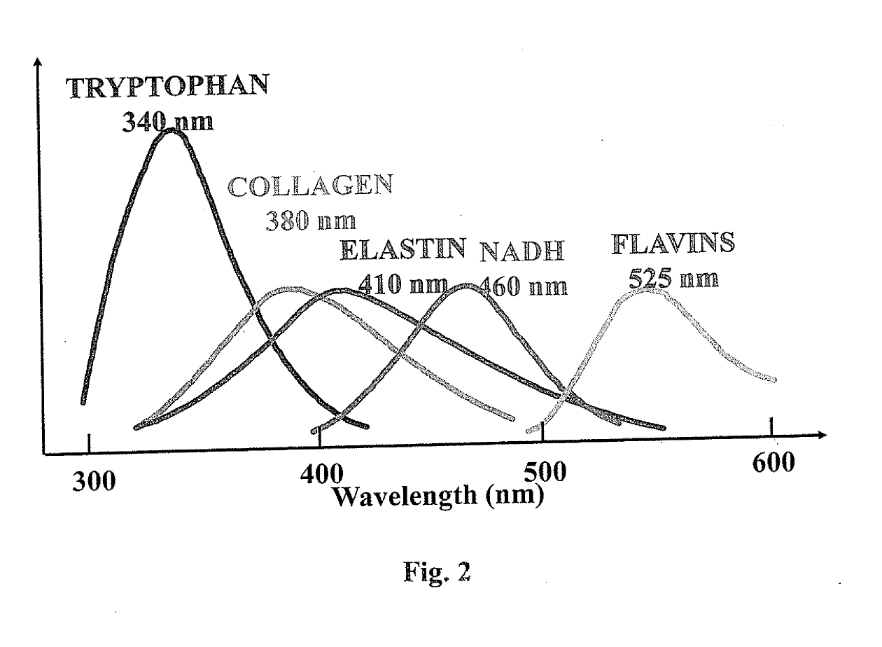

The present invention is directed to novel methods, techniques and systems, which can be used for detecting metastasis competence of cancerous cells. Referring to FIG. 1, the specific wavelengths were selected to investigate the key fluorophores related to cancer cells metastasis competence. The specific excitation wavelengths centered at 300 nm were selected to monitoring the content changes of fingerprint fluorophore for tryptophan at 300 nm most of the flurophanes of interest absorb substantially at the same levels. Referring to FIG. 2 the emission spectra of five of these tissue components are illustrated, showing the relative emission intensities and spectral distributions indicating the staggered or offsets in the wavelengths peaks.

The illumination light from laser diodes, or white lamp and light emission diodes with specific band pass wavelength. The specific long pass filter is in front of the sample side to ensure that the desired emission signals are recorded. By measuring the change of fluorescence profiles of food in different condition, one can sensitively monitor in real time the relative tryptophan content of different types of cells and determine the metastasis competence. The present invention uses native fluorescence spectroscopy, which is faster and easier compared to conventional diagnostic methods and can be built into small sized, light-weight and low cost devices and systems.

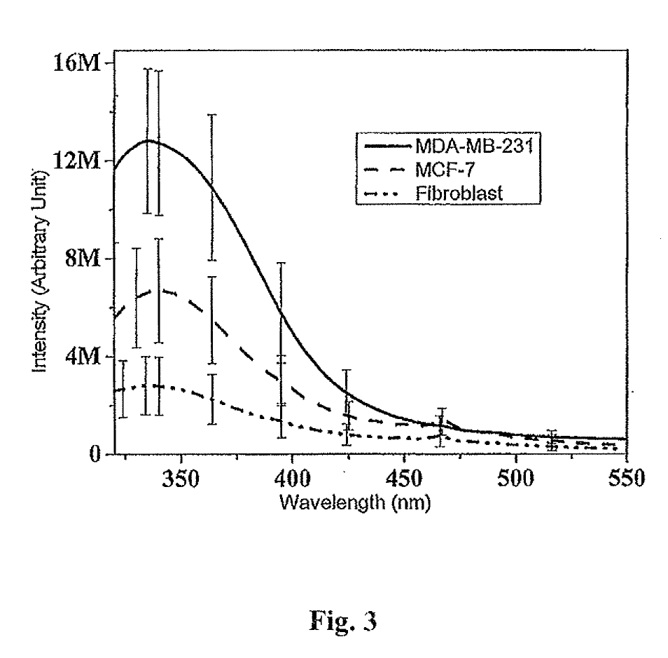

Referring now to NFL emission spectra from 3 types of cells in FIG. 3. The average fluorescence spectral profiles in the range of 320 nm to 580 nm of the fibroblast cells (n=16), MCF-7 cells (n=1.7) and MDA-MB-231 cells (n=18), measured by 300 nm excitation wavelength, are displayed in the FIG. 1 as solid, dash and dot lines, respectively. It shows that the emission spectra have consistency for the same types of cells but significant differences exist among these three types of cells. There are two peaks, at 340 nm and near 460 nm for all types of cells. The stronger peak occurs at 340 nm, which is known as the emission peak of tryptophan, and the weaker peak at 460 nm, which is known as the characteristic emission peak of NADH. The major difference of the profiles between MDA-MB-231 and MCF-7 or fibroblastic cell is that MDA-MB-231 was observed as having much higher fluorescence intensity at the peak of 340 nm than MCF-7 and fibroblastic cells have. It is also noticeable that the values of the peak near 460 nm for all types of cells are close to each other, and this peak for MCF-7 cells is slightly higher than the peak for MDA-MB-231 cells. As the cell density of each sample is well controlled and the value of intensity is normalized, the peaks indicate the contents of tryptophan and NADH from a given number of cells. From this profile, we found the MDA-MB-231 cells has a higher contents of tryptophan than that in MCF-7 cells or fibroblast cells, but has similar contents or even slightly less contents of NADH than that in other two types of cells.

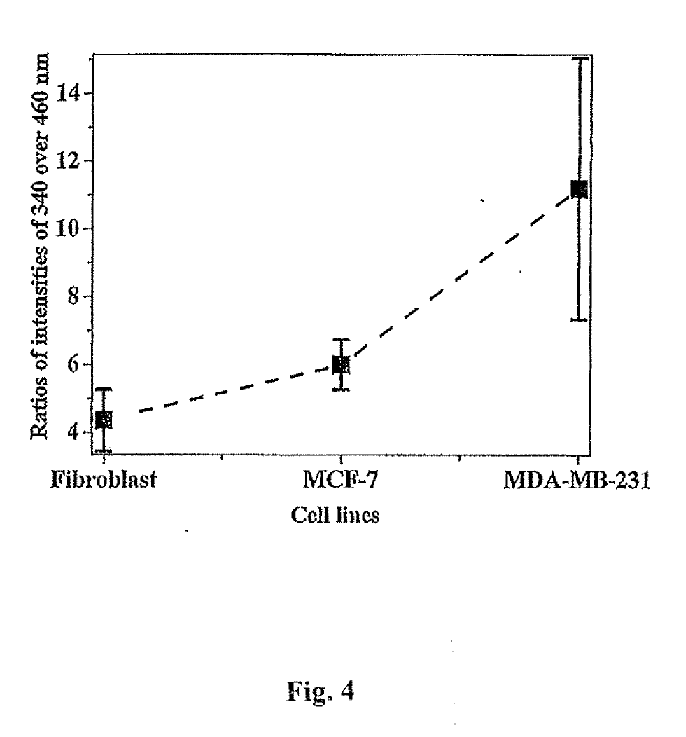

Referring now to the increase of average ratio R from non-aggressive to aggressive cancerous cells, and from normal cells to non-aggressive cancerous cells, FIG. 4 provides an alternate diagnostic criterion for detecting metastasis competence level. In order to evaluate this potential, FIG. 4 shows the calculated R (box) of the ratios as a function of normal, non-aggressive and aggressive cancerous cells. It is important to note that R exhibits a monotonous growth and a good correlation with the metastasis competence, average R=4.4 for normal cells, average R=5.9 for non-aggressive cancer cells and average R=11.2 for aggressive cancer cells. The linear increase of the R. as the function of metastasis competence reflects higher 340 over 460 nm ratio and contained a higher grade of aggressive cancerous cells in comparison with the lower grade non-aggressive cancerous cells as well as normal cells. This information can be summarized in the following table.

Ratio of 340 nm Over Different Reference Signals

| Fluorophones | | | | |
|---|---|---|---|---|
| Compound | Ratio | MDA-MB-231 | MCF-7 | Fibroblast |
| Tryptophan/NADH | I₃₄₀/I₄₆₀ | 11.2 | 5.9 | 4.4 |
| Tryptophan/NADH | I₃₄₀/I₅₀₀ | 16 | 10 | 7.7 |
| & Flavins | | | | |
| Tryptophan/Flavins | I₃₄₀/I₅₃₀ | 19.8 | 15.4 | 11.8 |

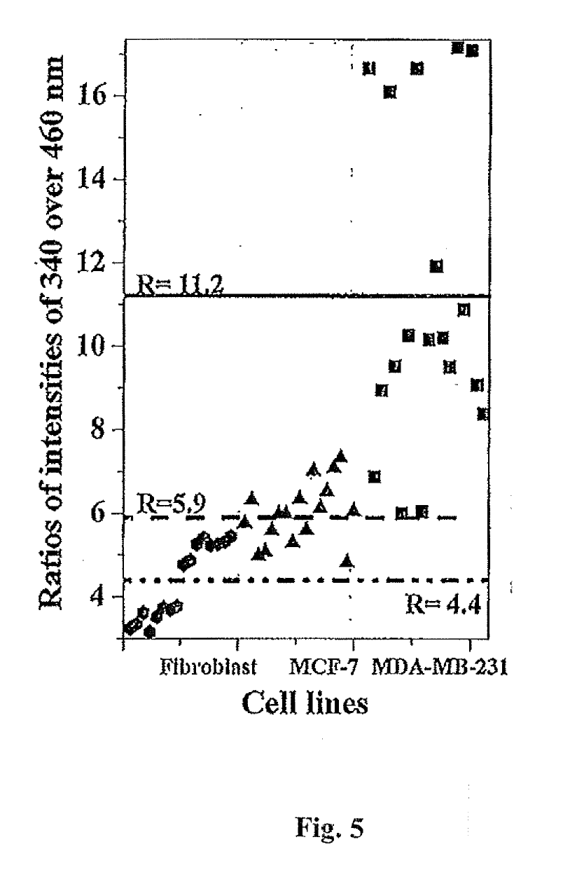

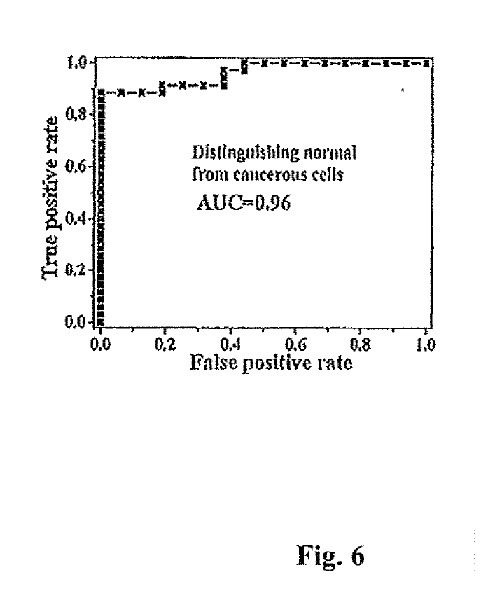

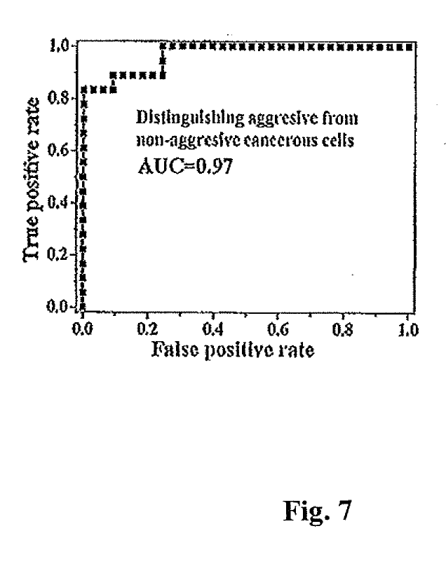

FIGS. 5-7 illustrate the relative tryptophan content of 3 types of cells and classification of metastasis competence in FIG. 3. Based on these observation, the ratios of intensities at 340 nm over 460 nm were calculated for each sample and compared the differences. The average ratio of all aggressive cancerous cells was 11.2, while the average ratio for non-aggressive cells were 5.9 and for normal cells the average ratio was 4.4. The average ratios are R₂₃₁>R_{mcF7}>R_{Normal}. The differences of ratios for 340 nm over 460 nm are prominently high in aggressive cancerous cells compared to non-aggressive cancerous cells as well as normal cells. There is good agreement with our past observations on breast tissue that used the ratio of 340 nm to 440 nm. Some cancer tissues have ratios above 10 and, even up to 50. In addition, these features were also demonstrated for aggressive prostate cancer cell.

NFL spectra have been used to diagnose the aggressiveness of breast cancer. This is the first time that cancer metastasis competence among aggressive cancer cells, non-aggressive cancer cells and normal cells was evaluated by measuring the relative contents of tryptophan using NFL. Our results demonstrate aggressive breast cancer cells have higher relative contents of tryptophan than other cells. Tryptophan is a good marker for aggressiveness of cancers using the ratio of 340 to 460 nm excited by 300 nm. This research indicates that measuring the NFL of tryptophan within the tissue can be used as a fingerprint for monitoring different metastatic cancers. A similar outcome was found in prostate cells supporting the results presented here. This technique may have potential to be used to monitor disease activity and response to therapy in cancer patients. From this research one can deduce that the magnitude of the ratio R from the tryptophan in cells indicates the degree on how aggressive a cancer is to metastasize, i.e. the greater the R the more aggressive the cancer and the more likely it will metastasize. Cells can be extracted from the organ (breast, prostate, brain, colon . . . ) and tested by R at 340/460 nm NSF to determine the level the aggressiveness of the tumor.

While NADH has been used in the examples as the reference fluorophore compared to tryptophan as the key biochemicals “fingerprint” other reference fluorophores within cells may also be used to provide and monitor relative emission spectra contents. For example flavin adenine dinucleotide or flavin (FAD) is another fluorophore found in cells and can also be used. In that case the ratios of intensities spectra need to be modified and should be taken at 340 nm (for tryptophan) over 525 nm (for FAD) where the emission spectra for flavins peak. However, regardless of the reference fluorophone(s) used the ratios R and results should be consistent since the levels of tryptophan will be the same and the resulting ratios R will continue to be useful in detecting the degree of metastasis competence of different cancer cells and the risk level of a cancer.

Any suitable clinically acceptable protocol for harvesting and isolating epithelial cells from tumor tissue cells for use in the method of the invention can be used. A revised method from one described in “Isolation of viable epithelial cells from human colons carcinoma tissue” by Tamas Micsik, Semmelweis University, Budapest, Hungary is given by way of example. A small piece of tumor tissue sample is placed into 15 ml vials containing RPMI 1640 cell culture media. The media is then removed and the tissue is transferred into Petri dishes and the samples are then cut into smaller pieces using a surgical blade. A sufficient amount of Hank's Balanced Salt Solution (HBSS) is placed on the cut pieces in order to avoid dehydration. After that, the tissue is transferred into 1.5 ml microfuge tubes, each containing 1,000 μl enzyme solutions of 14 Wünsch Unit activities of Liberase Dl and DH Research Grade. Vortex briefly and incubate in a moving/vibrating water bath at +37° C. for 10 minutes, or gently vortex vials several times during incubation. Add 200 μl of 10% fetal bovine serum to block the reaction. In order to prepare the cell suspension, and use a 70 μm mesh-cell filter to filter the mixture. Then add 1 ml of HBSS, with the tip of the pipettor pointing at the remainder solid tissue on the filter. Finally, harvest the cells by centrifugation at 2,000× g for 1 minute at +4° C. after decanting the supernatant. Before counting the cells re-suspend the pellet in an adequate amount of HBSS. Add trypan blue solution to 10 μl of the suspension, mix thoroughly, and allow standing for 5 minutes. Then transfer 10 μl of the trypan blue cell suspension to a hemocytometer and count the viable cells. An adequate amount of HBSS should be added to achieve a final volume of 4.2 ml (7×600 μl) cell suspension containing 1.4 to 3.5 million (7×200,000 to 500,000) viable cells.

It will be obvious to those skilled in the art that numerous changes and modifications can be made after reading, the above descriptions. Hence, the Claims attached should be construed to cover all the possible changes and modifications covered by the spirit and scope of this invention. Any and all equivalent contents and ranges in the Claims should be regarded as coming within the scope of this invention.

### REFERENCES

1. S. Gout and J. Huot, “Role of cancer microenvironment in metastasis: focus on colon cancer,” Cancer Microenviron 1(1). 69-83 (2008).

2. R. R. Alfano, D.Tata, J.Cordero, P. Toinashefsky, F. Longo, M. Alfano, “Laser induced fluorescence spectroscopy from native cancerous and normal tissue,” IEEE J Quantum Electron 20(1507-1511 (1984).

3. R. R. Alfano, B. B. Das, J. Cleary, R. Prudente and E. J. Celmer, “Light sheds light on cancer-distinguishing malignant tumors from benign tissues and tumors,” NY Acad Med 67(2), 143-150 (1991).

4. Y. P. Lin. Zhang, Jianpeng Xue, Sebastiao Pratavieira, Baogang Xu, Samuel Achilefu, R. R Alfano, “Tiyptophan as the key fingerprint for distinguishing aggressiveness among cancer cell lines using native fluorescence spectroscopy,” found of Biomedical Optics (submitted 2013).

5. R. Kim, M. Erni and K. Tanabe. “Cancer immunoediting from immune surveillance to immune escape,” Immunology 121(1), 1-14 (2007).

6. G. C. Prendergast, “Cancer: Why tumours eat tryptophan,” Nature 478(7368), 192-194 (2011).

What is claimed:

1. A method used for comparing relative levels of tryptophan to other fluorophores in cells by detecting emission spectra comprising the steps of illuminating the cells from patients or any other sources with excitation light having selective specific wavelengths ≦300 nm to excite tryptophan and other reference fluorophores; measuring emission intensity levels of tryptophan and of other reference fluorophores; and comparing said emission intensity of tryptophan at least one reference fluorophore.

2. A method as defined in claim 1, wherein the excitation light has characteristic wavelengths at approximately 300 nm to monitor the relative content change of tryptophan to other reference fluorophores, from the fluorescence emission spectral range from 320 nm to 580 nm; and determining a ratio of fluorescence intensities at about 340 nm to 460 nm.

3. A method as defined in claim 1, wherein comparing comprises forming a ratio R of tryptophan to reference fluorophore intensities.

4. A method as defined in claim 3, wherein said intensities are measured at 340 nm for tryptophan and at 440-460 nm for a reference fluorophore NADH: or Flavins at 525 nm.

5. A method as defined in claim 1, further comprising extracting cells from human or animal tissue selected from the group comprising lesion, tumor or growth from brain, breast, colon, oral cavity, liver, kidney, skin, vagina, cervix, prostate and measuring the fluorescence spectra excited with ≦300 nm to form an emission Ratio of fluorescence intensity R over a range for tryptophan and a reference fluorophore NADH from about 340 to 460 nm to determine the degree of cancer aggressiveness of the extracted cells.

6. A method as defined in claim 2, for detecting degree of metastasis competence of different cancer cells to diagnose the risk level of cancer comprising the steps of measuring the emission spectra of cells from patients or any other sources from tryptophan; and comparing the emission from a reference fluorophore NADH from 320 nm to 580 nm excited by 300 nm; and establishing the aggressiveness of cancer cells when an emission intensity ratio R of fluorescense of tryptophan to NADH is >10.

7. A method as defined in claim 1, wherein illumination for absorption is pumped within the range of 270-290 nm.

8. A method as defined in claim 3, used for distinguishing cancer cells from normal cells in diagnosis comprising the steps of measuring the emission spectra of cells from patients or any other source from tryptophan and comparing emission intensities from a reference fluorophore NADH and flavins from 320 nm to 580 nm excited by 300 nm; and establishing the ratio R for cancer cells when >6 R is greater than approximately 6.

9. A method as defined in claim 1, used for diagnosing whether cancer is positive or negative, malignant or benign comprising the steps of measuring the emission spectra of cells from patients or any other sources from key fluorophore tryptophan; comparing the emission from other reference fluorophore NADH from 320 nm to 580 nm excited by 300 nm and establishing that the cells are normal cells when ratio R<5.

10. A method as defined in claim 6, wherein an aggressive cancer is determined when said ratio R>11.

11. A method as defined in claim 3, used for detecting metastasis competence, further comprising the steps of tracking during therapy and treatment of cancer; measuring the emission spectra of cells from patients or any other sources, a key fluorophore tryptophan; comparing the emission from a reference fluorophore NADH from 320 nm to 580 nm excited at approximately 300 nm; tracking the average ratio R of the cells from patients; and determining if the treatments take effect.

12. A method as defined in claim 3, used for detecting metastasis competence for cancer cells and other normal cells in diagnosis further comprising the steps of using a Support Vector Machine (SVM) or Support Component Machine (SCM) to extract a criteria R used to set a diagnosis standard for distinguishing the aggressive cancer cells from non-aggressive cancer cells; and evaluating the metastasis level of the cancer.

13. A method as defined in claim 3, further comprising the step of using a Support Component (SC) to extract a criteria R used to set a diagnosis standard for distinguishing, the non-aggressive cancer cells from normal cells to evaluate the metastasis level of the cancer.

14. A method as defined in claim 1, wherein the cells are illuminated with selective absorption wavelengths to excite key fluorophore tryptophan at wavelengths approximately centered at 300 nm; and monitoring the content change of tryptophan by its emission spectra.

15. A method as defined in claim 3, further comprising the step of establishing a relative content ratio R of tryptophan over other reference fluorophore NADH or Fl.avins excited by 300 nm in the range from 320 nm to 580 nm; and calculating an average ratio R from different cells with the higher values of R indicating higher metastasis competence of cells.

16. The method as defined in the claim 1, wherein naturally occurring fingerprint fluorophore tryptophan a higher level of uptake by aggressive cancer cells than by non-aggressive cells or normal cells, is selected to determine the metastasis competence of cancer by the selective excitation wavelength centered at 300 nm at 320 nm to 580 nm from tryptophan.

17. The method as defined in claim 3, wherein a Support Vector Machine (SVM) is used to extract a criteria R to set a diagnosis standard for distinguish the aggressive cancer cells from non-aggressive cancer cells to evaluate the metastasis level of the cancer.

18. The method as defined in claim 2, wherein a mobile phone APP is used to analyze data, and to detect the metastasis competence or track effect of treatment for cancer.

19. The method as defined in claim 1, further comprising (a) monitoring cancer progress on patients;
(a) obtaining cell sample from the patients or animals with cancer by inserting a needle to extract a small number of cells; separating the cancer cells from normal cells; and growing a culture of the cells to an amount sufficient to diagnose;
(b) illuminating the cultured cell samples with light having selective wavelength to excite the key fluorophore tryptophan with another reference key fluorophore NADH, said light wavelength being centered at 300 nm to monitor the relative content of tryptophan to NADH; and
(c) establishing the relative content of tryptophan relative to NADH to indicate the metastasis competence of the cancer cells using fluorescence.

20. The method as defined in claim 1, further comprising the step of forming images of resultant fluorescence emitted from the cells sample extracted from the patients and selected from a group comprising of breast colon, brain, kidney, liver , cervix, vagina, skin, prostate or any other body part sources, or from a sample of cultured cells from a patients.

21. The method as defined in claim 1, wherein fluorescent molecules of tryptophan are selected as the key biochemically interpretable ‘fingerprints’ to monitor its relative content, and wherein the fluorescent molecule NADH is selected as a reference fluorophore combined with tryptophan to monitor tryptophan relative content, which reflects the metastasis competence of cancer.

22. A system for comparing relative levels of tryptophan to other fluorophores in cells by detecting emission spectra comprising means for illuminating the cells from patients or any other sources with excitation light having selective specific wavelengths ≦300 nm to excite tryptophan and other fluorophores; means for measuring emission intensity levels of tryptophan and intensity of tryptophan with a measured intensity of other reference fluorophores; and means for comparing said emission intensity of tryptophan with a measured intensity of at least one reference fluorophore.
